(19) United States
(12) Patent Application Publication
(10) Pub. No.: US 2019/0078240 A1
(43) Pub. Date: Mar. 14, 2019

(54) THREE DIMENSIONAL MEDICAL WOVEN FABRIC CONFIGURATION

(71) Applicant: KIPAS MENSUCAT ISLETMELERI ANONIM SIRKETI, Kahramanmaras (TR)

(72) Inventor: Gökhan TANDOGAN, Kahramanmaras (TR)

(21) Appl. No.: 16/106,385

(22) Filed: Aug. 21, 2018

(30) Foreign Application Priority Data
Sep. 14, 2017 (TR) 2017/13573

(51) Int. Cl. D03D 1/00; D03D 11/02; D03D 25/00; B32B 5/02; B32B 5/26; B32B 3/28; D06M 15/643; A61L 31/08; A61L 31/16

(52) U.S. Cl. CPC D03D 1/00; D06M 2101/32; D03D 25/005; B32B 5/024; B32B 5/26; B32B 3/28; D06M 15/643; A61L 31/08; A61L 31/16; D03D 2700/0111; D10B 2331/04; D10B 2509/00; D10B 2201/02; D10B 2401/022; D03D 11/02

(57) ABSTRACT

A medical fabric obtained by a rapier loom such, having an upper fabric layer, a lower fabric layer and a hollow part defined between the lower fabric layer and the upper fabric layer in such a way to form a three dimensional structure in order to decrease the pressure exerted on the body parts of patients. The fabric has a dimensional surface which allows the upper fabric layer to form a textured structure by defining the hollow part due to the tension exerted on the lower fabric layer at the end of the loom.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### 1. Field of the Invention

The invention relates to a medical fabric configuration having a three dimensional structure to gain air permeability and probiotic property.

The invention in particular relates to a three dimensional woven fabric configuration intended to be used reduce the risk of developing pressure ulcers, having high air permeability and obtained using double layered fabric manufacturing technique.

### 2. Description of Related Art Including Information Disclosed Under 37 CFR 1.97 and 37 CFR 1.98.

Pressure ulcers, also known as bedsores, are skin and muscle lacerations seen in patients who have been on bed-rest for a long time. The continuous pressure exerted on certain locations of the body of patients who has been on bed rest for a long time slows down the blood circulation and leads to necrosis and tissue damage in the related part of the body. Today, pressure ulcers are commonly encountered problems which lead to physical and psychological distress experienced both by the health institutions and the patients and patient relatives. In particular, treatment of pressure ulcers once they are formed is a long and painful process. Therefore, a variety of medical products are have been developed to prevent pressure ulcers.

The developed medical products include a variety of products such as mattresses, bed covers, mattress protectors, bedsheets etc. These products are expected to possess a number of features such as being antistatic, providing dryness, allow high air permeability without liquid permeability, supporting blood circulation, being antiallergenic, providing thermal insulation etc. Therefore, technologies that have been developed in the recent years accelerated development of fabric and nonwoven surfaces that will be able to provide these qualities.

Three dimensional fabric structures called spacers are used between structures with pressure ulcer prevention features in the medical sector. In addition to fabric structures obtained through warp knitting technology, structures made of nonwoven surfaces are also used. Application no. CN201670924 discloses the medical use of a double layer fabric produced using a double rapier weaving machine to prevent pressure ulcers. The mentioned invention provides a three dimensional structure with ventilation holes using binding yarn between the fabrics obtained as two layers. Use of binding yarn in this invention constitutes an extra operational process, increases fabric weight and therefore leads to increased costs in unit length of fabric.

Therefore, lack of a solution to the aforementioned problems in the prior art have made it necessary to make a development in the relevant technical field.

### BRIEF SUMMARY OF THE INVENTION

The present invention relates to a three dimensional medical fabric configuration obtained through double layer fabric manufacturing technology intended to be used to reduce the risk of developing pressure ulcers in order to overcome the aforementioned disadvantages and provide further advantages in the relevant technical field.

The primary object of the invention is to provide a medical fabric, obtained by means of rapier loom, which exhibit increased load bearing capacity and high air permeability with its three dimensional structure, in order to reduce the risk of developing pressure ulcers.

Another object of the invention is to provide a medical fabric with probiotic property.

Another object of the invention is to provide a medical fabric with increased washing resistance.

In order to achieve all of the aforementioned objects and those will be inferred form the detailed description given below, a medical fabric, obtained by means of a rapier loom, comprising an upper fabric layer, a lower fabric layer and a hollow part defined between the said lower fabric layer and the upper fabric layer in such a way to form a three dimensional structure in order to decrease the pressure exerted on the body parts of patients to prevent developing pressure ulcers, is provided. Accordingly, the mentioned medical fabric comprises a dimensional surface which allows the upper fabric layer to form a textured structure by defining the said hollow part due to the tension exerted on the lower fabric layer at the end of the loom.

A preferred embodiment of the invention comprises first weft yarns and first warp yarns formed according to a draft plan obtained by drafting with a weaving repeat comprising plied yarns in order to define the said dimensional surface of the said upper fabric layer.

Another preferred embodiment of the invention comprises second weft yarns and second warp yarns formed according to 1/1 weaving repeat such that the said lower fabric layer defines a flat surface.

In another preferred embodiment of the invention, the said first weft yarns and first warp yarns are Ne 50/2 pes and 78 dtex elastane blended elastane polyester.

In another preferred embodiment of the invention, the said weft yarns and second warp yarns are Ne 50/2 cotton.

In another preferred embodiment of the invention, a probiotic chemical application process is applied to the said medical fabric.

In another preferred embodiment of the invention, 15-30 g/lt Ulpraphil HSD is used in the probiotic chemical application process as the hydrophilic silicone.

In another preferred embodiment of the invention, 5 g/lt BIO DC is used in the probiotic chemical application process as the probiotic chemical finishing material.

In another preferred embodiment of the invention, 0.5 g/lt Securan TA is used in the probiotic chemical application process as the pH stabilizer.

In another preferred embodiment of the invention, the probiotic chemical application process is conducted using stenters at 100-120° C. and according to padding principle.

The configuration and further elements of the invention with their advantages shall become apparent with the drawing explained below, therefore, assessment should be made based on these drawings.

### BRIEF DESCRIPTION OF THE SEVERAL VIEWS OF THE DRAWINGS

FIG. 1 is a schematic view of the cross section of the medical fabric of the invention.

FIG. 2 illustrates the drafting-weaving repeat regarding the medical fabric.

### REFERENCE NUMBERS

10 Medical Fabric
11 Upper Fabric Layer
111 Dimensional Surface
112 First Weft Yarn
113 First Warp Yarn
12 Hollow Part
13 Lower Fabric Layer
131 Flat Surface

### DETAILED DESCRIPTION OF THE INVENTION

The innovation of the invention shall become apparent with the examples given by the herein detailed description without limiting the scope of the invention.

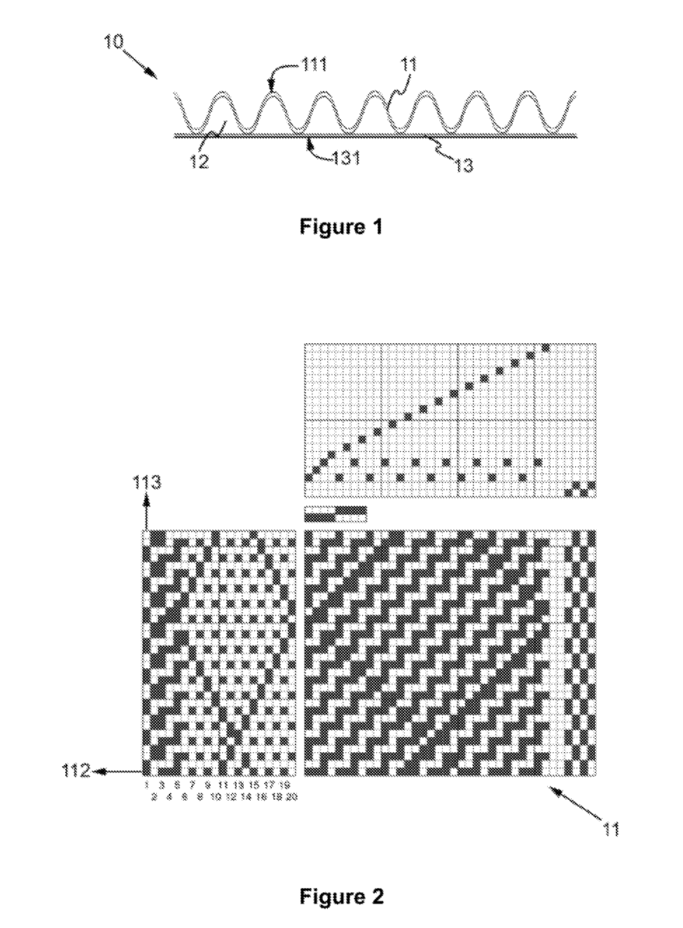

The medical fabric (10), whose cross section view is given in FIG. 1, mainly comprises an upper fabric layer (11) and a lower fabric layer (13), and further comprises a hollow part (12) defined between the said upper fabric layer (11) and the lower fabric layer (13). The upper fabric layer (11) defines a one dimensional surface. In order to form the said dimensional surface (111), the upper fabric layer (11) comprises a first weft yarn (112) and a first warp yarn (113). The lower fabric layer (13) defines a flat surface (131). The lower fabric layer (13) comprises a second weft yarn and a second warp yarn such that it forms the said flat surface (131). In order to form the mentioned fabric structure, the weaving repeat and the draft repeat based on the weaving draft given in FIG. 2 is used.

The medical fabric (10) of the invention is formed at a draft rapier loom with a double layer form using a single loom beam. At the upper fabric layer (11), both the first weft yarn (112) and the first warp yarn (113) are made of same the raw material, which is cotton (combed) yarn Ne 50/2 made of spun fiber yarn. On the lower fabric layer (13), both the second weft yarn and the second warp yarn are made of the same raw material, which is Ne 50/2 pes+78 dtex elastane blended yarn. These yarns also act as binding yarn and used with 1/1 repeat with intervals. Yarn counts may vary between Ne 20/1 and Ne 60/1. Weight of the obtained medical fabric (10) is 300-400 gr/m².

The strains exerted on cotton and polyester/elastane yarns during forming the medical fabric (10) lead to different impacts in terms of the recovery mechanism between the upper fabric layer (11) and the lower fabric layer (13). It both provides the contraction via recovery of the elastane polyester yarns and the lower fabric layer (13) due to the 1/1 weaving repeat. Since the weaving repeat applied to the first weft yarn (112) and the first warp yarn (113) forming the upper fabric layer (11) is different from 1/1 weaving repeat, the rigidity of the obtained upper fabric layer (11) is also substantially different from the rigidity of the lower fabric layer (13). Moreover, recovery behavior of the cotton yarn is lower than that of the elastane. Therefore, contraction of the lower fabric layer (13) leads to forming the dimensional surface (111) of the upper fabric layer (11). This structure forms the hollow part (12) containing air between the lower fabric layer (13) and the upper fabric layer (11), while at the same time transforming the medical fabric (10), which is woven two in a two dimensional form, into a three dimensional form. Moreover, forming the dimensional surface (111) on the upper fabric layer (11) is also affected by the texture surface as a result of using double plied yarns.

In order to prevent deterioration of the 3 dimensional structure, free processes are applied instead of cylindrical printed processes during the curing and finishing processes applied to the raw state of the medical fabric (10). In order to prevent deterioration of the concave form achieved on the fabric during the curing and finishing processes, it is important to avoid cylindrical printing process such as sanforizing printing or calendering processes. During the curing and finishing processes, washing-drying-fixing-dyeing/optics-washing-drying-chemical finishing processes are applied. The dimensional surface (111) of the upper fabric layer (11) of the medical fabric (10) reduces to pressure exerted on the patient's body in order to prevent developing pressure ulcers.

In order to make the obtained medical fabric (10) gain probiotic property, chemical application process is performed. While the antibacterial chemical treatment removes the beneficial and harmful bacteria entirely, the mentioned probiotic chemical application allows only the harmful bacteria to be removed from the obtained fabric while preserving the beneficial bacteria. Therefore, wound healing process is supported with the existence of beneficial bacteria in case of an open wound. The probiotic chemical treatment applied therefor is performed by applying a water based solution containing hydrophilic silicones and probiotic microorganisms to the medical fabric (10) using stenters at 100-120° C. depending on the padding method. Through the application, the amount of the chemical finishing material within the body of the medical fabric (10) varies between 3-10 g/lt according to CFU/cm2 (Bio) levels.

The recipe of the solution used in the chemical process is:
15-30 g/lt Ultraphil HSD as the hydrophilic silicone
5 g/lt BIO DC as the probiotic chemical finishing material
0.5 g/lt Securan TA as the pH stabilizer.

With the medical fabric (10) of the invention, a fabric with high wash resistance, high air permeability with its textured structure, high load bearing capability and probiotic property is obtained. These features allow the medical fabric (10) to provide an alternative to the three dimensional woven fabrics used especially for treatment of pressure ulcers. Furthermore, using this medical fabric (10) in upholstery industry along with the sponge structures, which are used to soften and provide volume to the floor surfaces, will allow less or thinner structures of these sponge structures to be used.

1. A medical fabric obtained by means of a rapier loom such, comprising an upper fabric layer, a lower fabric layer and a hollow part defined between the said lower fabric layer and the upper fabric layer in such a way to form a three dimensional structure in order to decrease the pressure exerted on the body parts of patients, wherein, it comprises a dimensional surface which allows the upper fabric layer to form a textured structure by defining the said hollow part due to the tension exerted on the lower fabric layer at the end of the loom.

2. A medical fabric according to claim 1, wherein; it comprises first weft yarns and first warp yarns formed according to a draft plan obtained by drafting with a weaving repeat comprising plied yarns in order to define the said dimensional surface of the said upper fabric layer.

3. A medical fabric according to claim 2, wherein; the said first weft yarns and first warp yarns are Ne 50/2 pes and 78 dtex elastane blended elastane polyester.

4. A medical fabric according to claim 1, wherein; it comprises second weft yarns and second warp yarns formed according to 1/1 weaving repeat such that the said lower fabric layer defines a flat surface.

5. A medical fabric according to claim 4, wherein; the said weft yarns and second warp yarns are Ne 50/2 cotton.

6. A medical fabric according to claim 1, wherein, a probiotic application process is applied to the said medical fabric.

7. A medical fabric according to claim 6, wherein, 15-30 g/lt Ulpraphil HSD is used in the probiotic chemical application process as the hydrophilic silicone.

8. A medical fabric according to claim 6, wherein, 5 g/lt BIO DC is used in the probiotic chemical application process as the probiotic chemical finishing material.

9. A medical fabric according to claim 6, wherein, 0.5 g/lt Securan TA is used in the probiotic chemical application process as the pH stabilizer.

10. A medical fabric according to claim 6, wherein, the probiotic chemical application process is conducted using stenters at 100-120° C. and according to padding principle.
